(19) United States
(12) Patent Application Publication
(10) Pub. No.: US 2020/0397717 A1
(43) Pub. Date: Dec. 24, 2020

(54) Hemp Oil / Magnesium Preparation for Cognitive-Enhancement and Reduction of Stress and Inflammation

(71) Applicant: Food Technology and Design LLC, BDA FoodPharma, Santa Fe Springs, CA (US)

(72) Inventor: Timothy McCoy Marshall, Tucson, AZ (US)

(21) Appl. No.: 16/673,125

(22) Filed: Nov. 4, 2019

Related U.S. Application Data
(60) Provisional application No. 62/864,974, filed on Jun. 21, 2019.

(51) Int. Cl. A61K 31/05; A61K 33/08; A61K 33/10; A61K 33/12; A61K 31/07; A61K 31/51; A61K 31/525; A61K 31/4415; A61K 31/375; A61K 31/593; A61K 9/00

(52) U.S. Cl. CPC A61K 31/05; A61K 33/08; A61K 33/10; A61K 33/12; A61K 31/07; A61K 9/0095; A61K 31/525; A61K 31/4415; A61K 31/375; A61K 31/593; A61K 31/51

(57) ABSTRACT

Novel neuroprotective preparations of hemp oil and magnesium are disclosed herein. Antioxidant and anti-inflammatory activity of hemp oil (containing a broad-spectrum of beneficial phytochemicals) are combined with magnesium and has been found to reduce oxidative stress, upregulation of antioxidant enzyme systems, and the mitigation of NMDA-receptor hyperactivity. The synergistic effects of hemp oil and bioavailable magnesium are utilized to improve cognitive function, mood, stress tolerance, and reduce oxidative stress and inflammation. A highly-efficacious, lipophilic, trans-mucosal, delivery system is included to provide exceptional bioavailability of a palatable and convenient intra-oral, or topical, anti-inflammatory, neurotrophic, and neuroprotective preparation. Additionally, methods of use are described, allowing for effective, safe, and convenient use for general (public) and clinical use, in both humans and animals.

### BACKGROUND

Unabated oxidative stress due to suboptimal, deficient intake of specific antioxidant nutrients (e.g. magnesium, lithium, zinc, selenium, molybdenum, boron, vitamins C, D, E) and plant-derived antioxidants (e.g. terpenes, phenolics, cannabinoids)—coupled with neuronal excitotoxicity and the associated increase in free-radical generation and inflammation—is a pathophysiological pathway common to a host of primary neurological conditions, neurodegenerative diseases, and secondary neurological conditions leading to diminished cognitive, memory, motor, and sensory function.

Some examples of these primary conditions include depression, anxiety, sleep disturbances, chronic pain, sensitivity to stress, and chronic inflammation. Examples of secondary conditions include traumatic brain injury (“TBI”), exposure to certain neurotoxins, and cerebral ischemia. All of these disease states share the following common physiological characteristics: oxidative stress from excessive unabated free-radical generation, elevated inflammatory biomarkers (e.g. hsCRP), excessive stimulation of post-synaptic neurons (N-methyl-D-aspartate receptor mediated; NMDA-R), primarily from the excitatory amino acid neurotransmitters, aspartate and glutamate, and chronic inflammation. Magnesium, zinc, and lithium deficiencies contribute to NMDA receptor hyperactivity, and to the overstimulation of the post-synaptic neuron leading to a long-standing partially depolarized state, which opens cell membrane calcium channels, allowing an influx of calcium ions and increasing intracellular calcium concentrations. Calcium is a cofactor for a plethora of intracellular enzymes, including proteases, DNA and RNA endonucleases, and phospholipases. Intracellular hypercalcemia resulting from overstimulation may lead to widespread activation of these enzymes causing destruction of cellular proteins, nucleic acids, and membrane structures resulting in eventual neuronal death.

Accordingly, what is needed is a neuroprotective, antioxidant, anti-inflammatory preparation for the prevention and addressment of a wide variety of primary and secondary health issues (e.g. cognitive decline, poor focus or concentration, sensitivity to stress, sleep disturbances) involving the brain and nervous system that is highly efficacious, non-toxic, and possesses high-bioavailability (easily absorbable into the systemic circulation) due to the high-lipophilicity of its therapeutic elements (e.g. essential minerals, phytochemicals). While many attempts have been made to address this need, arriving at compositions that fulfill many if not all of the objectives above remains elusive.

### SUMMARY OF THE DISCLOSURE

The embodiments herein relate to highly-bioavailable, novel, neurotrophic and neuroprotective preparations comprising a combination of hemp oil—containing beneficial, naturally-occurring phytocompounds (i.e. phytochemicals; e.g. terpenes, phenolics, cannabinoids)—combined with magnesium and a proprietary blend of synergistic vitamins and essential trace minerals for enhanced antioxidant, anti-inflammatory, mood-enhancing, and stress-reducing benefits. In particular, the embodiments herein relate to preparations of hemp oil containing a full-spectrum of beneficial, nontoxic, phytochemicals in combination with nominally-ionized, highly-stable, highly-bioavailable, lipophilic, mineral chelates (e.g. glycinates) of magnesium and other essential minerals—in a proprietary neuroprotective blend for highly-efficient, oral (e.g. sublingual, gingival, soft palatal, buccal absorption) and topical delivery, and methods of use in humans and animals.

An optimal, therapeutic neuroprotective intervention to prevent and/or treat unabated oxidative stress and post-synaptic overstimulation would ideally create a microenvironment wherein oxidative stress is reduced and the depolarization threshold for opening calcium channels in the cell membranes of neurons is increased. Additionally, preventive therapy must be convenient for use—and easy to take—making a gummy or “soft-chew”, chewable tablet, lozenge, quick-dissolve tablet, “chewing gum”, liquid spray, liquid beverage, liquid “shot”, tincture, “food-form,” partially orally-absorbable preparation greatly advantageous.

Topical creams, lotions, salves, balms, “patches”, and other external methods of application could also be employed to deliver the neuroprotective, antioxidant, anti-inflammatory preparation consisting of a full-spectrum of beneficial phytochemicals and cannabinoids (hemp oil), bioavailable magnesium, and proprietary blend of synergistic nutrients (e.g. vitamins, minerals) to the skin. Hence, in some embodiments, the novel, highly-bioavailable, hemp oil/magnesium neuro-protectant preparations, as described herein, could also be employed for a wide range of skin conditions (e.g. acne, eczema, dermatitis, psoriasis) known to have chronic inflammation, as a common, underlying component. Cannabinoids and the essential mineral magnesium are known to have antioxidant and anti-inflammatory properties both independent of, and dependent on NMDA receptor inhibition, respectively. Cannabinoids exert their beneficial effects through their innate free-radical scavenging (antioxidant) activity, cannabinoid receptor activation, and epigenetic modulation whereas magnesium exerts its influence through NMDA receptor inhibition and multi-modal, synergistic interactions with a wide-variety of vitamins (e.g. vitamin D, B-complex), minerals (e.g. lithium, zinc), and the upregulation of endogenous, antioxidant enzyme systems.

In view of the above, some embodiments relate to cognitive-enhancing, stress-reducing, antioxidant, anti-inflammatory preparations containing hemp oil with a full-spectrum of beneficial phytochemicals and highly-bioavailable, nominally-ionized, lipophilic magnesium coupled with selected, synergistic vitamins and highly-bioavailable, nominally-ionized, lipophilic, essential trace elements (i.e., micronutrients). Other embodiments relate to hemp oil—containing a broad-spectrum of beneficial terpenes, phenolics, and cannabinoids—in combination with highly bioavailable magnesium as the 2-component core, enhanced with additional synergistic nutrients in a next-generation, antioxidant, anti-inflammatory neuroprotective preparation for cognitive enhancement (e.g. memory, mood, focus, concentration), stress reduction, and a wide-variety of health and skin benefits.

Also disclosed is a full-spectrum cannabinoid preparation from hemp oil, coupled with magnesium and a proprietary blend of synergistic nutrients delivered in a highly-efficacious, lipophilic, oral (and topical) delivery system.

In some embodiments, the neuroprotective preparation further comprises a hemp extract (i.e., hemp oil) containing a full-spectrum of naturally-occurring phytochemicals (e.g., terpenes, phenolics, cannabinoids) obtained from the hemp plant. In some embodiments, the hemp oil is derived from organically-grown hemp. In some embodiments, the hemp oil is derived from conventionally-grown hemp.

In some embodiments, the neuroprotective preparation further comprises a magnesium chelate. In some embodiments, the magnesium chelating compound is chosen from the group consisting of succinic acid, ascorbic acid, aspartic acid, threonic acid, lysinic acid, malic acid, tauric acid, citric acid, and gluconic acid. In some embodiments, the magnesium chelating compound is a salt of orotic acid. In some embodiments, the magnesium chelate is a salt of glycine.

In this disclosure, “about” means within +/−10% of a stated value.

In some embodiments, the amount of cannabidiol (CBD) is between about 0.50 and 50.0 milligrams per serving.

In some embodiments, the amount of elemental magnesium is between about 10 and 500 milligrams per serving.

### DETAILED DESCRIPTION OF EMBODIMENTS

As discussed above, the disclosed embodiments relate to preparations of hemp oil containing a full-spectrum of naturally-occurring phytochemicals (e.g. terpenes, phenolics, cannabinoids) obtained from the hemp plant in combination with highly-bioavailable, nominally-ionized, lipophilic magnesium (e.g., Mg-bisglycinate) and a proprietary neurosupportive, antioxidant, anti-inflammatory blend of highly-bioavailable, nominally-ionized, lipophilic trace minerals (e.g., Zn-bisglycinate) and other synergistic nutrients for oral (e.g. gingival, buccal, soft palatal, sublingual) delivery and absorption as a central nervous system neurotrophic and neuroprotectant and topical administration for reducing local pain and inflammation, and methods of use.

A wide range of primary central neurological diseases and secondary conditions manifest cognitive, memory, motor, and sensory impairment as primary and debilitating symptoms. A few non-limiting examples of such diseases and conditions are listed herein above and include amyotrophic lateral sclerosis (“ALD”), Parkinson's Disease (“PD”), Alzheimer's Disease (“AD”), post-traumatic stress disorder (“PTSD”), attention deficit hyperactivity disorder (“ADHD”), depression/anxiety, and tinnitus. Examples of secondary conditions include traumatic brain injury (“TBI”), chronic post-traumatic or post-surgical neuropathic pain, acute or chronic exposure to certain toxins, for example mercury, lead, cadmium, arsenic, polychlorinated biphenyls, and ethanol; and cerebral ischemia.

Cannabinoids (from hemp oil) and magnesium exert a myriad of antioxidant, anti-inflammatory benefits on the brain and nervous system, cardiovascular system, heart, liver, kidneys, skin, and for general health benefits.

Magnesium exerts a portion of its neuroprotective, antioxidant, anti-inflammatory effects through inhibition of the N-methyl-D-aspartate receptor (“NMDA”), with which magnesium (and/or lithium, zinc) interact as a receptor ligand, on post-synaptic cortical neurons of the central nervous system. NMDA receptors are ubiquitous throughout the brain and play a role in regulation of the excitatory state of post-synaptic neurons. NMDA receptors act as a cationic membrane “pore,” primarily for calcium ions although other cations such as sodium, zinc, and protons may pass into the cell. In conditions wherein the post-synaptic neuron is polarized and glutamate is absent from the synapse, a local negative membrane charge permits the pore to be blocked with a magnesium ion. Under conditions wherein 1) glutamate is present within the synapse at a sufficient concentration; and 2) the post-synaptic neuron is partially depolarized creating a neutral or relative positive membrane charge, the magnesium ion is displaced, the pore opens, and calcium ions are allowed to pass freely through the NMDA receptor into the cell. Once intracellular, calcium exerts a myriad of secondary effects, largely through its role as a secondary messenger and enzyme cofactor. Increased intracellular calcium leads to increased cellular enzyme activity of proteases, nucleases, and phospholipases, breaking down structural components and functional machinery of the cell and often leading to cell death.

Keeping the baseline state of the NMDA receptor pore in a closed configuration, therefore, is important for the proper function and survival of a post-synaptic neuron. There are at least two potential sites of action to keep the receptor pore closed to influx of calcium and other cations into the neuron: 1) adequate-to-high synaptic magnesium concentrations; and 2) tyrosine-mediated phosphorylation of the NR2B receptor subunit.

Given a polarized or neutral post-synaptic cell membrane, in combination with adequate extracellular magnesium concentrations, magnesium binds to the receptor pore and blocks influx of calcium.

Several physiologic mechanisms resist a partially depolarized state in the post-synaptic cell membrane, keeping the pore closed to the influx of calcium an enhancing appropriate NMDA receptor function. One of these potentiating mechanisms is tyrosine-mediated phosphorylation of the NMDA receptor subunits, tending to “close” the receptor pore by causing an amphoteric shift in one or more protein subunits. Tyrosine phosphatase-mediated NR2B subunit phosphorylation potentiated by the lithium cation has been shown to cause depression of NMDA receptor currents.

The present embodiments seek to militate against activation of a final common pathway for neuronal cell injury and death—elevated intracellular calcium levels—by impeding permeability of the NMDA receptor to calcium.

Magnesium concentrations in a neuronal synapse are necessary to saturate the post-synaptic population of NMDA receptors, therein keeping the receptor pores closed to the influx of extracellular calcium ions when the post-synaptic neuron is in a partially polarized state. Hypomagnesaemia is associated with a plethora of symptomatic neurological abnormalities, such as depression, anxiety, sleep disturbances, hyperreflexia, tremor, confusion, hallucinations, convulsions, hyperacusis, nystagmus, tetany, delirium tremens, and extrapyramidal disorders.

Trans-mucosal absorption of elemental magnesium is greatly increased by combining elemental magnesium with a stable, nominally-ionized, organic chelate. In some embodiments, the magnesium chelating compound is a salt of glycine. In some embodiments, the magnesium chelating compound is a salt of orotic acid. In some embodiments, the magnesium chelating compound is a salt of succinic acid, aspartic acid, ascorbic acid, threonic acid, gluconic acid, lysinic acid, malic acid, tauric acid, or citric acid. It is anticipated that as experimentation and research in the art of enhanced trans-mucosal magnesium absorption progresses, other chelating compounds may be used as a chelating compound, in some embodiments.

The total concentration of elemental magnesium per dose or serving of the neuroprotective preparation is calculated to provide adequate intracerebral levels of magnesium without being so high as to increase the risk of toxicity from hypermagnesaemia. Accordingly, the amount of elemental magnesium, in some embodiments, is between approximately 10 milligrams and 400 milligrams.

Proprietary neurosupportive blend, in some embodiments, is a commercially available blend of vitamins, minerals, and other nutrients compounded to reduce oxidative stress, inflammation, and promote neurological and general health. In some embodiments, the proprietary neurosupportive blend comprises (per serving): magnesium as glycinate, malate, or taurinate (as well as other organic or inorganic forms of the mineral), 10-500 mg; kelp, 5-100 mg; vitamin A as retinyl palmitate or acetate or beta-carotene, 500-10,000 iu; vitamin B1, 0.5-100 mg; vitamin B2, 0.5-100 mg; vitamin B6, 0.5-100 mg; Vitamin B3, 0.5-250 mg; vitamin B5, 5-200 mg; Vitamin B12, 5-5,000 mcg as cyanocobalamin or methylcobalamin or hydroxocobalamin or adenosylcobalamin; folic acid or methyl-folate or folinic acid, 10-1,000 mcg; 25-1,000 mcg; non-GMO vitamin C, 25-1,000 mg; vitamin D3, 500-10,000 IU; vitamin E as d-alpha tocopherol or dl-alpha tocopheryl and/or mixed tocopherols and/or tocotrienols, 10-400 iu for alpha-tocopherol and 5-500 mg for mixed tocopherols including gamma-tocopherol or the family of tocotrienols; boron as glycinate (as well as other organic or inorganic forms of the mineral), 0.5-10 mg; copper as bisglycinate (as well as other organic or inorganic forms of the mineral), 0.1-5 mg; zinc as bisglycinate (as well as other organic or inorganic forms of the mineral), 2.5-50 mg; manganese as bisglycinate (as well as other organic or inorganic forms of the mineral), 0.5-10 mg; selenium as glycinate (as well as other organic or inorganic forms of the mineral), 10-400 mcg; molybdenum as glycinate (as well as other organic or inorganic forms of the mineral), 10-400 mcg; and chromium as nicotinate glycinate (as well as other organic or inorganic forms of the mineral), 10-1,000 mcg.

The delivery system in some embodiments is a highly specialized combination of the most bioavailable nutrients (e.g. Albion® minerals; kelp (Icelandic, Norwegian, or Organic), a rich source of iodine and essential trace minerals; Extramel French melon extract supplying SOD/Catalase) along with non-GMO tableting agents and excipients which facilitate oral bioavailability through mucosal absorption from within the oral cavity. Some non-limiting examples of such excipient compounds include xylitol, erythritol, allulose, cellulose, palm oil, coconut oil, silicon dioxide, citric acid, malic acid, organic stevia (leaf) extract, monk fruit extract, and natural flavors.

Other such compounds as are known in the art of oral drug absorption and delivery systems may be used. In these and some other embodiments, the neuroprotective preparation is taken as a tablet, soft chew, gummy, chewable wafer, quick-dissolve tablet, or lozenge placed in the mouth. In some embodiments, the lozenge is held under the tongue or placed in a buccal recess and allowed to dissolve. In some embodiments, the soft chew is chewed, allowing exposure of the phytochemicals (e.g. terpenes, phenolics, cannabinoids), highly-bioavailable, lipophilic, magnesium, and other highly-bioavailable (ie. stable, nominally-ionized, lipophilic), essential trace elements and synergistic nutrients to the microvascular tissue (e.g. gingival, buccal, soft palatal, and sublingual surfaces) of the oral cavity for efficient oro-mucosal transport and absorption of the nutrients, and swallowed.

A neuroprotective, antioxidant, anti-inflammatory preparation and method of use has been described. Based upon known mechanisms for unabated oxidative stress (e.g. nutrient deficiencies, suboptimal dietary antioxidant consumption), chronic inflammation, and neuronal cell death secondary to excitotoxicity—the neuroprotective preparation acts to decrease oxidative stress through the innate antioxidant activity of a full-spectrum of terpenes, phenolics, and cannabinoids (from hemp oil), the upregulation of endogenous antioxidant enzyme systems (e.g. superoxide dismutase, SOD; glutathione peroxidase, GPx; catalase), and to militate against development of increased post-synaptic neuronal intracellular calcium levels by acting upon (via modulation and inhibition) NMDA receptors in the post-synaptic cell membrane. The synergistic effects of a broad-spectrum cannabinoid (hemp) extract (i.e. hemp oil) and magnesium are effectively coupled to a proprietary blend of synergistic, antioxidant, anti-inflammatory nutrients to create an optimal therapeutic effect with no undesirable, adverse effects (i.e. side-effects).

### Non-Limiting Example

In a study conducted at a neurology clinic in Tucson, Ariz., volunteers received a dose (equivalent to 4 soft chews supplying 200 mg of magnesium (from malate, taurinate) and 5.0 mg of CBD (from 50 mg of organic European hemp oil extract). This trial assessed overall effectiveness in terms of a self-reported greater sense of well-being.

A scientifically validated General Well-Being Survey was given to participants before and after taking the “HempMag:” supplement. Study participants reported “having no muscle tension, I can feel at this moment” (having previously reported neck tightness), feeling “better, calm, and clear-headed,” “I feel completely calm. No stress. Focus seems to be a lot better,” “no pain, left shoulder injury/inflammation is no longer there after taking supplement,” “overall sensation is good/great. I feel like I can take on the world, “I noticed I was more focused and clear-headed,” and similar responses that indicated a calming, anti-inflammatory effect. The embodiments and examples set forth herein were presented to enable those of ordinary skill in the art to make and use said embodiments. However, those of ordinary skill in the art will recognize that the foregoing description and examples have been presented for the purposes of illustration and example only. The description as set forth is not intended to be exhaustive or to limit the claims to the precise form disclosed. Many modifications and variations are possible in light of the teachings above.

What is claimed is:

1. A composition comprising hemp oil having a cannabidiol (CBD) content of about between 0.50 and 50.0 milligrams per serving and magnesium in an amount of about 10-500 milligrams per serving.

2. The composition of claim 1, wherein the hemp oil comprises a full-spectrum of naturally-occurring phytochemicals obtained from a hemp plant.

3. The composition of claim 1, wherein said magnesium is a magnesium chelate.

4. The composition of claim 1, wherein said magnesium is an inorganic magnesium salt.

5. The composition of claim 3, wherein the magnesium chelate comprises a counter-ion or a chelating compound selected from the group consisting of succinic acid, ascorbic acid, aspartic acid, threonic acid, lysinic acid, malic acid, tauric acid, citric acid, and gluconic acid.

6. The composition of claim 3, wherein the magnesium chelate comprises a chelating compound of a salt of orotic acid or a salt of glycine.

7. The composition of claim 4, wherein the inorganic magnesium salt comprises a counter-ion selected from the group consisting of carbonate, chloride, oxide, and sulfate.

8. The composition of claim 1, wherein the magnesium is elemental magnesium.

9. The composition of claim 1, wherein the magnesium is provided as malate, glycinate, taurinate, or threonate.

10. The composition of claim 1, further including: kelp, 5-100 mg; vitamin A as retinol or retinyl palmitate or acetate or beta-carotene, 500-10,000 iu; vitamin B1, 0.5-100 mg; vitamin B2, 0.5-100 mg; vitamin B6, 0.5-100 mg; Vitamin B3, 0.5-250 mg; vitamin B5, 5-200 mg; Vitamin B12, 5-5,000 mcg as cyanocobalamin or methylcobalamin or hydroxocobalamin or adenosylcobalamin; folic acid or methyl-folate or folinic acid, 10-1,000 mcg; 25-1,000 mcg; non-GMO vitamin C, 25-1,000 mg; vitamin D3, 500-10,000 IU; vitamin E as d-alpha tocopherol or dl-alpha tocopheryl and/or mixed tocopherols and/or tocotrienols, 5-400 iu for alpha-tocopherol and 5-500 mg for mixed tocopherols including gamma-tocopherol or the family of tocotrienols; boron as glycinate (as well as other organic or inorganic forms of the mineral), 0.5-10 mg; copper as bisglycinate (as well as other organic or inorganic forms of the mineral), 0.1-3 mg; zinc as bisglycinate (as well as other organic or inorganic forms of the mineral), 2.5-50 mg; manganese as bisglycinate (as well as other organic or inorganic forms of the mineral), 0.5-5 mg; selenium as glycinate (as well as other organic or inorganic forms of the mineral), 10-200 mcg; molybdenum as glycinate (as well as other organic or inorganic forms of the mineral), 10-400 mcg; and chromium as nicotinate glycinate (as well as other organic or inorganic forms of the mineral), 10-1,000 mcg.

11. The composition of claim 2, further including: kelp, 5-100 mg; vitamin A as retinol or retinyl palmitate or acetate or beta-carotene, 500-10,000 iu; vitamin B1, 0.5-100 mg; vitamin B2, 0.5-100 mg; vitamin B6, 0.5-100 mg; Vitamin B3, 0.5-250 mg; vitamin B5, 5-200 mg; Vitamin B12, 5-5,000 mcg as cyanocobalamin or methylcobalamin or hydroxocobalamin or adenosylcobalamin; folic acid or methyl-folate or folinic acid, 10-1,000 mcg; 25-1,000 mcg; non-GMO vitamin C, 25-1,000 mg; vitamin D3, 500-10,000 IU; vitamin E as d-alpha tocopherol or dl-alpha tocopheryl and/or mixed tocopherols and/or tocotrienols, 5-400 iu for alpha-tocopherol and 5-500 mg for mixed tocopherols including gamma-tocopherol or the family of tocotrienols; boron as glycinate (as well as other organic or inorganic forms of the mineral), 0.5-10 mg; copper as bisglycinate (as well as other organic or inorganic forms of the mineral), 0.1-3 mg; zinc as bisglycinate (as well as other organic or inorganic forms of the mineral), 2.5-50 mg; manganese as bisglycinate (as well as other organic or inorganic forms of the mineral), 0.5-5 mg; selenium as glycinate (as well as other organic or inorganic forms of the mineral), 10-200 mcg; molybdenum as glycinate (as well as other organic or inorganic forms of the mineral), 10-400 mcg; and chromium as nicotinate glycinate (as well as other organic or inorganic forms of the mineral), 10-1,000 mcg.

12. The composition of claim 3, further including: kelp, 10-100 mg; vitamin A as retinol or retinyl palmitate or acetate or beta-carotene, 500-10,000 iu; vitamin B1, 0.5-100 mg; vitamin B2, 0.5-100 mg; vitamin B6, 0.5-100 mg; Vitamin B3, 0.5-250 mg; vitamin B5, 5-200 mg; Vitamin B12, 5-5,000 mcg as cyanocobalamin or methylcobalamin or hydroxocobalamin or adenosylcobalamin; folic acid or methyl-folate or folinic acid, 10-1,000 mcg; 25-1,000 mcg; non-GMO vitamin C, 25-1,000 mg; vitamin D3, 500-10,000 IU; vitamin E as d-alpha tocopherol or dl-alpha tocopheryl and/or mixed tocopherols and/or tocotrienols, 5-400 iu for alpha-tocopherol and 5-500 mg for mixed tocopherols including gamma-tocopherol or the family of tocotrienols; boron as glycinate (as well as other organic or inorganic forms of the mineral), 0.5-10 mg; copper as bisglycinate (as well as other organic or inorganic forms of the mineral), 0.1-3 mg; zinc as bisglycinate (as well as other organic or inorganic forms of the mineral), 2.5-50 mg; manganese as bisglycinate (as well as other organic or inorganic forms of the mineral), 0.5-5 mg; selenium as glycinate (as well as other organic or inorganic forms of the mineral), 10-200 mcg; molybdenum as glycinate (as well as other organic or inorganic forms of the mineral), 10-400 mcg; and chromium as nicotinate glycinate (as well as other organic or inorganic forms of the mineral), 10-1,000 mcg.

13. The composition of claim 4, further including: kelp, 10-100 mg; vitamin A as retinol or retinyl palmitate or acetate or beta-carotene, 500-10,000 iu; vitamin B1, 0.5-100 mg; vitamin B2, 0.5-100 mg; vitamin B6, 0.5-100 mg; Vitamin B3, 0.5-250 mg; vitamin B5, 5-200 mg; Vitamin B12, 5-5,000 mcg as cyanocobalamin or methylcobalamin or hydroxocobalamin or adenosylcobalamin; folic acid or methyl-folate or folinic acid, 10-1,000 mcg; 25-1,000 mcg; non-GMO vitamin C, 25-1,000 mg; vitamin D3, 500-10,000 IU; vitamin E as d-alpha tocopherol or dl-alpha tocopheryl and/or mixed tocopherols and/or tocotrienols, 5-400 iu for alpha-tocopherol and 5-500 mg for mixed tocopherols including gamma-tocopherol or the family of tocotrienols; boron as glycinate (as well as other organic or inorganic forms of the mineral), 0.5-10 mg; copper as bisglycinate (as well as other organic or inorganic forms of the mineral), 0.1-3 mg; zinc as bisglycinate (as well as other organic or inorganic forms of the mineral), 2.5-50 mg; manganese as bisglycinate (as well as other organic or inorganic forms of the mineral), 0.5-5 mg; selenium as glycinate (as well as other organic or inorganic forms of the mineral), 10-200 mcg; molybdenum as glycinate (as well as other organic or inorganic forms of the mineral), 10-400 mcg; and chromium as nicotinate glycinate (as well as other organic or inorganic forms of the mineral), 10-1,000 mcg.

14. The composition of claim 5, further including: kelp, 10-100 mg; vitamin A as retinol or retinyl palmitate or acetate or beta-carotene, 500-10,000 iu; vitamin B1, 0.5-100 mg; vitamin B2, 0.5-100 mg; vitamin B6, 0.5-100 mg; Vitamin B3, 0.5-250 mg; vitamin B5, 5-200 mg; Vitamin B12, 5-5,000 mcg as cyanocobalamin or methylcobalamin or hydroxocobalamin or adenosylcobalamin; folic acid or methyl-folate or folinic acid, 10-1,000 mcg; 25-1,000 mcg; non-GMO vitamin C, 25-1,000 mg; vitamin D3, 500-10,000 IU; vitamin E as d-alpha tocopherol or dl-alpha tocopheryl and/or mixed tocopherols and/or tocotrienols, 5-400 iu for alpha-tocopherol and 5-500 mg for mixed tocopherols including gamma-tocopherol or the family of tocotrienols; boron as glycinate (as well as other organic or inorganic forms of the mineral), 0.5-10 mg; copper as bisglycinate (as well as other organic or inorganic forms of the mineral), 0.1-3 mg; zinc as bisglycinate (as well as other organic or inorganic forms of the mineral), 2.5-50 mg; manganese as bisglycinate (as well as other organic or inorganic forms of the mineral), 0.5-5 mg; selenium as glycinate (as well as other organic or inorganic forms of the mineral), 10-200 mcg; molybdenum as glycinate (as well as other organic or inorganic forms of the mineral), 10-400 mcg; and chromium as nicotinate glycinate (as well as other organic or inorganic forms of the mineral), 10-1,000 mcg.

15. The composition of claim 6, further including: kelp, 10-100 mg; vitamin A as retinol or retinyl palmitate or acetate or beta-carotene, 500-10,000 iu; vitamin B1, 0.5-100 mg; vitamin B2, 0.5-100 mg; vitamin B6, 0.5-100 mg; Vitamin B3, 0.5-250 mg; vitamin B5, 5-200 mg; Vitamin B12, 5-5,000 mcg as cyanocobalamin or methylcobalamin or hydroxocobalamin or adenosylcobalamin; folic acid or methyl-folate or folinic acid, 10-1,000 mcg; 25-1,000 mcg; non-GMO vitamin C, 25-1,000 mg; vitamin D3, 500-10,000 IU; vitamin E as d-alpha tocopherol or dl-alpha tocopheryl and/or mixed tocopherols and/or tocotrienols, 5-400 iu for alpha-tocopherol and 5-500 mg for mixed tocopherols including gamma-tocopherol or the family of tocotrienols; boron as glycinate (as well as other organic or inorganic forms of the mineral), 0.5-10 mg; copper as bisglycinate (as well as other organic or inorganic forms of the mineral), 0.1-3 mg; zinc as bisglycinate (as well as other organic or inorganic forms of the mineral), 2.5-50 mg; manganese as bisglycinate (as well as other organic or inorganic forms of the mineral), 0.5-5 mg; selenium as glycinate (as well as other organic or inorganic forms of the mineral), 10-200 mcg; molybdenum as glycinate (as well as other organic or inorganic forms of the mineral), 10-400 mcg; and chromium as nicotinate glycinate (as well as other organic or inorganic forms of the mineral), 10-1,000 mcg.

16. The composition of claim 1, further including one or more of a kelp, a French melon extract or concentrate, and a blueberry extract or concentrate.

17. The composition of claim 1, further including a tableting agent and an excipient that facilitates oral bioavailability through mucosal absorption from within the oral cavity.

18. The composition of claim 17, wherein said excipient that facilitates oral bioavailability through mucosal absorption from within the oral cavity comprises one or more of tapioca syrup, isomalto-oligosaccharide (IMO) syrup, powdered isomalto-oligosaccharide (IMO), honey, powdered honey, yacon syrup, agave syrup, corn syrup, glucose syrup, coconut sugar syrup, coconut sugar, date syrup, molasses, rice syrup, sugar cane syrup, raw cane sugar, cane sugar syrup, turbinado syrup, allulose syrup, maltitol syrup, polyglycitol syrup, sugar beet syrup, inulin syrup, powdered inulin, fibrosol, maltodextrin, dextrin, gum arabic, dextrose anhydrous, dextrose monohydrate, dried glucose syrup, sorghum syrup, tagatose syrup, and the following sugar alcohols: erythritol syrup, mannitol syrup, sorbitol syrup, or xylitol syrup, ethylene glycol, glycerol, erythritol, threitol, arabitol, xylitol, ribitol, mannitol, sorbitol, galactitol, fucitol, iditol, and inositol combined with any combination of the following: palm oil, coconut oil, citric acid, malic acid, fumaric acid, tartaric acid, soy and/or sunflower lecithin, silicon dioxide, cellulose, stevia (leaf) extract, monk fruit extract, natural or artificial flavors, saccharin, acesulfame, aspartame, neotame, and sucralose.
